(19) United States
(12) Patent Application Publication
(10) Pub. No.: US 2025/0389529 A1
(43) Pub. Date: Dec. 25, 2025

(54) TIME DOMAIN COHERENCE TOMOGRAPHY SYSTEM AND METHOD BASED ON PHOTONIC INTEGRATED CHIP

(71) Applicant: SILITH TECHNOLOGY (SHANGHAI) CO., LTD., Shanghai (CN)

(72) Inventors: Guangzhu ZHOU, Shanghai (CN); Zhexuan ZHENG, Shanghai (CN); Rui ZHU, Shanghai (CN); Senlin ZHANG, Shanghai (CN); Yuxuan CHEN, Shanghai (CN)

(73) Assignee: SILITH TECHNOLOGY (SHANGHAI) CO., LTD., Shanghai (CN)

(21) Appl. No.: 19/023,449

(22) Filed: Jan. 16, 2025

(30) Foreign Application Priority Data
Jun. 20, 2024 (CN) 202410796240.0

(51) Int. Cl. G01B 9/02091; G01B 9/02015

(52) U.S. Cl. CPC G01B 9/02091; G01B 9/02027

(57) ABSTRACT

By arranging light source, optical chip, interferometer, photoelectric detection module, sample acquisition module and data processing module, wherein light source is connected to interferometer by optical chip, while both reference path and sample path of optical chip have optical delay line arranged respectively, and optical delay lines are continuously adjustable; optical chip is configured to divide detection light into reference path signal light and sample path signal light, and transmitting reference path signal light and sample path signal light to interferometer by optical delay line continuously adjustable respectively. Comparing with traditional mechanical style TD-OCT system having problem of scanning rate slow, present solution replaces a mechanical movement of reference path in prior art by optical delay lines continuously adjustable, which not only improves scanning rate, but also has smaller volume.

### CROSS-REFERENCE TO RELATED APPLICATION

This application claims the priority benefit of China application serial no. 202410796240.0, filed on Jun. 20, 2024. The entirety of the above-mentioned patent application is hereby incorporated by reference herein and made a part of this specification.

### BACKGROUND

### Technical Field

The present application relates to the technical field of optical coherence tomography, and in particular to a time domain coherence tomography system and method based on a photonic integrated chip.

### Description of Related Art

An Optical Coherence Tomography (OCT) is a medical imaging technology, has been widely applied in a plurality of applications including human eye non-invasive imaging and more. OCT is an optical test method based on low coherence interference, which obtains a plurality of deep-level information of a sample, having achieved a high-resolution in axial by an interference between a reference path signal and a backscattering signal of a sample path.

In an OCT system, an optical coupler divides a detection light emitted by a light source into two paths, one path is referred to as a reference path, and another path is a sample path. A light in the reference path passes through a delay path, while a light in the sample path is directed to a sample to be detected by a front-end optical system. A photoelectric detector finally receives the light reflected by the sample and the light coming from the reference path, while an intensity of an interference signal between the light reflected by the sample and the light coming from the reference path reflects a reflectivity information on a specific depth of the sample to be detected.

In a Time Domain OCT (TD-OCT) imaging system, in order to achieve scanning a sample in a depth direction (referred to as an A-Scan), it requires to introduce a time-variable optical path length or an optical delay amount to the reference path, which is generally achieved by a reflector moving mechanically. However, due to a mechanical component itself and an inertial characteristic thereof, there is a problem existing that, a scanning rate which a traditional TD-OCT system is able to achieve, has been greatly limited (typically it can reach only a few hundred Hz), and such a mechanical scheme has a system volume relatively large and a cost high, which is not conducive to device miniaturization.

Therefore, a time domain coherence tomography system and a method based on a photonic integrated chip is needed urgently to improve the problem stated above.

### SUMMARY

The purpose of the present application is providing a time domain coherence tomography system and method based on a photonic integrated chip, being able to improve the scanning rate while reducing the system volume.

On a first aspect, the present application provides a time domain coherence tomography system based on a photonic integrated chip, comprising a light source, an optical chip, an interferometer, a photoelectric detection module, a sample acquisition module and a data processing module, wherein the light source is connected to the interferometer by the optical chip, while each of both a reference path and a sample path of the optical chip has an optical delay line arranged respectively, and the optical delay lines are continuously adjustable;
the light source is configured to emit a detection light;
the optical chip is configured to divide the detection light into a reference path signal light and a sample path signal light, and transmit the reference path signal light and the sample path signal light to an interferometer by an optical delay line continuously adjustable respectively;
the interferometer is configured to transmit the reference path signal light to generate a reference signal, before transmitting the reference signal to the photoelectric detection module; and configured to transmit the sample path signal light to a sample to be scanned to generate a reflection signal, before transmitting the reflection signal to the photoelectric detection module;
the photoelectric detection module is configured to receive the reference signal and the reflection signal, before generating an electrical signal after an interference;
the sample acquisition module is configured to receive the electrical signal and transmit the electrical signal to the data processing module;
the data processing module is configured to process the electrical signal to obtain an image of the sample to be scanned.

A beneficial effect of the present application is: by arranging a light source, an optical chip, an interferometer, a photoelectric detection module, a sample acquisition module and a data processing module, wherein the light source is connected to the interferometer by the optical chip, while both a reference path and a sample path of the optical chip have an optical delay line arranged respectively, and the optical delay lines are continuously adjustable; the optical chip is configured to divide the detection light into a reference path signal light and a sample path signal light, and transmit the reference path signal light and the sample path signal light to an interferometer by an optical delay line continuously adjustable respectively. Comparing with a traditional mechanical style TD-OCT system having the problem of the scanning rate slow, the present application replaces a mechanical movement of the reference path in the prior art by the optical delay lines continuously adjustable, which not only improves the scanning rate, but also has a smaller volume.

Preferably, the optical chip comprises a silicon substrate, a buried oxide layer, a silicon nitride waveguide layer, and a metal electrode.

Preferably, the optical chip comprises an optical input port formed by a first edge coupler, a light splitter, a reference path, a sample path, a first output port formed by a second edge coupler, and a second output port formed by a third edge coupler; the reference path and the sample path having an first optical delay line continuously adjustable and a second optical delay line continuously adjustable arranged respectively and symmetrically;
after having been input by the first edge coupler, the detection light is divided into the reference path signal light and the sample path signal light by the light splitter, while the reference path signal light is output by the first output port, and the sample path signal light is output by the second output port.

Preferably, the optical delay line is of a spiral structure; and/or the reference path of the optical chip further has a link arranged, the link is formed by an N+1-level cascaded optical switch and an N-level cascaded optical delay line, while an optical delay amount of each level of the optical delay line is fixed but different; wherein N is a positive integer;
the reference path signal light passes through the first optical delay line continuously adjustable and passes through the link, before being output by the first output port;
the sample path signal light passes through the second optical delay line continuously adjustable, before being output by the second output port directly.

Preferably, a length of the optical delay line on each level of the N-level cascaded optical delay line with a fixed delay amount growing exponentially, while a delay amount of a next-level optical delay line is greater than a delay amount of a previous-level optical delay line; and/or the fixed delay amount of a first-level optical delay line is less than or equal to a maximum delay amount of the optical delay line continuously adjustable.

Preferably, the first output port and the second output port of the optical chip are coupled through a bi-channel optical fiber array, and the sample path signal light is collimated by an optical fiber collimator.

Preferably, the optical switch is a 2×2 type adiabatic directional coupler or a multimode interferometer.

Preferably, the system further comprises a control module, configured to adjust a delay amount of the first optical delay line continuously adjustable and a delay amount of the second optical delay line continuously adjustable; and configured to control the optical switch on each level to adjust a total optical delay amount; after determining a target delay amount and selecting a plurality of optical switches to generate a delay most matching the target delay amount, together with the delay amount of the first optical delay line continuously adjustable and the delay amount of the second optical delay line continuously adjustable, a relative delay amount between two paths is locked accurately to the target delay amount.

Preferably, the light source is coupled to the optical chip through an optical fiber in a coupling manner of a coupler or a lens.

Preferably, the photoelectric detection module is arranged between the interferometer and the sample acquisition module;
the photoelectric detection module comprises a photoelectric detector and a transimpedance amplifier;
the photoelectric detector is configured to receive a signal of the reference path signal light having been transmitted through the interferometer as the reference signal, and receive an echo signal generated by the sample to be scanned as the reflection signal; and configured to generate a current signal after an interference between the reference signal and the reflection signal, and transmit the current signal to the transimpedance amplifier;
the transimpedance amplifier is configured to generate a voltage signal correspondingly according to the current signal, and transmit the voltage signal to the sample acquisition module.

Preferably, the system further comprises a scanning element, and the scanning element comprises a two-dimensional rotating mirror;
the scanning element is configured to guide the sample path signal light emitted by the interferometer to the sample to be scanned through the two-dimensional rotating mirror, so as to generate a plurality of echo signals at a plurality of different positions of the sample to be scanned, before completing extracting the information on a specific depth level of the sample to be scanned.

Preferably, the light source is a low coherence broadband light source; and/or the interferometer is a Michelson interferometer.

On a second aspect, the present application further provides a time domain coherence tomography method based on a photonic integrated chip, comprising:
a light source emitting a detection light;
an optical chip dividing the detection light into a reference path signal light and a sample path signal light, and transmitting the reference path signal light and the sample path signal light to an interferometer by an optical delay line continuously adjustable respectively;
the interferometer transmitting the reference path signal light to generate a reference signal, then transmitting the reference signal to a photoelectric detection module; and transmitting the sample path signal light to a sample to be scanned to generate a reflection signal, then transmitting the reflection signal to the photoelectric detection module;
the photoelectric detection module receiving the reference signal and the reflection signal, and generating an electrical signal after an interference;
a sample acquisition module receiving the electrical signal and transmitting the electrical signal to a data processing module;
the data processing module processing the electrical signal to obtain an image of the sample to be scanned.

A beneficial effect of the second aspect may refer to the description of the first aspect.

### BRIEF DESCRIPTION OF THE DRAWINGS

FIG. 1 illustrates a schematic structural diagram on a time domain coherence tomography system based on a photonic integrated chip according to an embodiment of the present application;

FIG. 2 illustrates a schematic structural diagram on an optical chip in the time domain coherence tomography system based on the photonic integrated chip according to an embodiment of the present application;

FIG. 3 illustrates a schematic structural diagram on a light interference link and a scanning frame in the time domain coherence tomography system based on the photonic integrated chip according to an embodiment of the present application;

FIG. 4 illustrates a schematic flowchart on a time domain coherence tomography method based on the photonic integrated chip according to an embodiment of the present application.

### DESCRIPTION OF THE EMBODIMENTS

In order to make the purpose, technical solution and advantages of the present application clearer and more explicit, further detailed descriptions of the present application are stated here, referencing to the attached drawings and some embodiments of the present application. Obviously, the described embodiments are part of, but not all of, the embodiments of the present application. Based on the embodiments of the present application, all other embodiments obtained by those of ordinary skills in the art without any creative work are included in the scope of protection of the present application. Unless otherwise defined, technical or scientific terms used herein should have the meanings usually understood by those of ordinary skills in the art to which the present application belongs. As used herein, the terms “comprise” and the like are intended to mean that an element or item appearing before the term encompasses elements or items appearing after the term and the equivalents thereof, instead of excluding other elements or items.

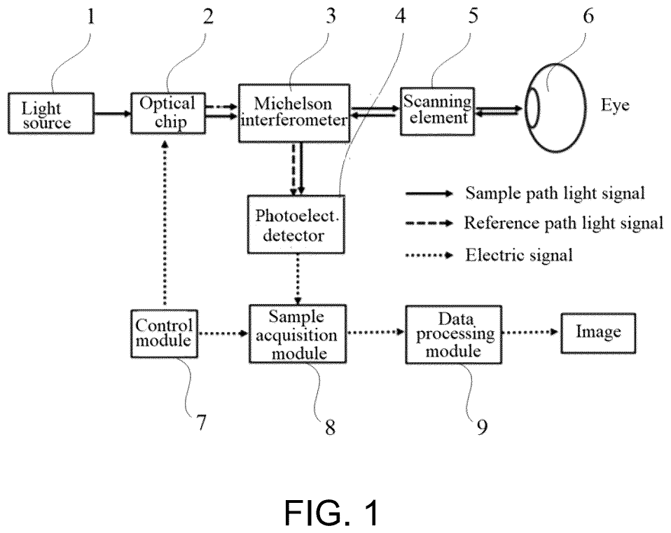

Shown as FIG. 1, the present application provides a time domain coherence tomography system based on a photonic integrated chip, comprising a light source 1, an optical chip 2, an interferometer, such as a Michelson interferometer 3, a photoelectric detection module, such as a photoelectric detector 4, a sample acquisition module 8 and a data processing module 9, wherein the light source 1 is connected to the interferometer by the optical chip 2, while each of both a reference path and a sample path of the optical chip 2 has an optical delay line arranged respectively, which is continuously adjustable; the light source 1 is configured to emit a detection light; the optical chip 2 is configured to divide the detection light into a reference path signal light and a sample path signal light, and transmit the reference path signal light and the sample path signal light to an interferometer by an optical delay line continuously adjustable respectively; the interferometer is configured to transmit the reference path signal light to generate a reference signal, before transmitting the reference signal to the photoelectric detection module; and configured to transmit the sample path signal light to a sample to be scanned, such as an eye 6, to generate a reflection signal, before transmitting the reflection signal to the photoelectric detection module; the photoelectric detection module is configured to receive the reference signal and the reflection signal, before generating an electrical signal after an interference; the sample acquisition module 8 is configured to receive the electrical signal and transmit the electrical signal to the data processing module 9; the data processing module 9 is configured to process the electrical signal to obtain an image of the sample to be scanned.

In a plurality of embodiments, the light source 1 is a low coherence broadband light source, typically, a super-radiation light emitting diode (SLD). In order to make an OCT imaging system have a high axial resolution, the light source 1 should have a coherence length relatively small. Accordingly, when an operating wavelength is fixed, the coherence length in turn determines a bandwidth of the light source 1 as desired. For a typical parameter of a wavelength 1060 nm, a coherence length should be in an order of microns.

In a plurality of embodiments, the light source 1 is coupled to the optical chip 2 through an optical fiber in a manner of an edge coupler or a lens coupling, and a light in the optical fiber is converted into a guided wave in a waveguide.

In a plurality of embodiments, the optical chip 2 comprises a silicon substrate, a buried oxide layer, a silicon nitride waveguide layer, and a metal electrode.

In a plurality of embodiments, the optical chip 2 comprises a first edge coupler 21 forming an optical input port, a light splitter, a reference path, a sample path, a first output port formed by a second edge coupler 22, and a second output port formed by a third edge coupler 23; the reference path and the sample path having an first optical delay line continuously adjustable and a second optical delay line continuously adjustable arranged respectively and symmetrically; after being input by the first edge coupler 21, the detection light is divided into the reference path signal light and the sample path signal light by the light splitter, while the first output port outputs the reference path signal light, and the second output port outputs the sample path signal light.

The optical input port of the optical chip 2 is connected to the light source 1 through an optical fiber. The light splitter divides an input light beam into two paths in a certain proportion (for example, 50:50), one path is the reference path, and another path is the sample path.

The sample path and the reference path each has the optical delay line arranged, which is precise and continuously adjustable, while two optical delay lines are arranged symmetrically, so as to cancel a fixed delay amount caused by a long delay line, and control a relative optical delay amount of two paths of signals. Preferably, the optical delay line has a layout of a spiral structure. A relative maximum delay amount generated by two paths of the delay lines in the present step is relatively small, which is unable to meet a millimeter-level large-scale detection depth required in an OCT application. Therefore, in a plurality of specific embodiments, the reference path of the optical chip 2 further has a link arranged, the link is formed by an N+1-level cascaded optical switch and an N-level cascaded optical delay line, while an optical delay amount of each level of the optical delay line is fixed but different; wherein N is a positive integer; the reference path signal light passes through the first optical delay line continuously adjustable before passing through the link, and being output by the first output port; the sample path signal light passes through the second optical delay line continuously adjustable, before being output by the second output port directly. The optical switch is a 2×2 type adiabatic directional coupler (ADC) or a multimode interferometer (MMI), one of two output ports of the coupler is input into a designed optical delay path, before being connected to one input port of a coupler in a next level, and another output port is directly connected to another input port of the coupler in the next level through a straight waveguide (having an extremely small delay amount), while selecting an output path is controlled by a thermo-optic effect of a silicon nitride waveguide.

In a plurality of other specific embodiments, a length of the optical delay line on each level of the N-level cascaded optical delay line having the fixed delay amount growing exponentially, while a delay amount of a next-level optical delay line is greater than a delay amount of a previous-level optical delay line (in an embodiment, L₁=L, L₂=2L, L₃=4L, . . . , Lₙ=2ⁿ⁻¹L), obtaining a corresponding optical delay amount by turning on/off a switch on each level to select different waveguide paths; and/or the fixed delay amount of a first-level optical delay line is less than or equal to a maximum delay amount of the optical delay line continuously adjustable.

In a plurality of specific embodiments, the first output port and the second output port of the optical chip 2 are coupled through a bi-channel optical Fiber Array (FA), and the sample path signal light is collimated by an optical fiber collimator 10. Then both the reference path signal light and the sample path signal light are transmitted to the Michelson interferometer 3 through the optical fiber, wherein the reference path signal light is received by the photoelectric detector 4 after passing through the Michelson interferometer 3, while the sample path signal light is guided to the scanning element 5 through the Michelson interferometer 3.

In a plurality of embodiments, the system further comprises a scanning element 5, and the scanning element 5 comprises a two-dimensional rotating mirror 51; the scanning element 5 is configured to guide the sample path signal light emitted by the Michelson interferometer 3 to the sample to be scanned through the two-dimensional rotating mirror 51, so as to generate a plurality of echo signals (that is, the echo signals are considered as the reflection signals) at a plurality of different positions of the sample to be scanned, before completing an extraction of the information on a specific depth level of the sample to be scanned. The echo signals are generated at different positions of the to-be-scanned sample (that is, the echo signal is applied as a reflection signal) to complete extracting the information on the specific depth level of the to-be-scanned sample.

In a plurality of embodiments, the photoelectric detection module is arranged between the Michelson interferometer 3 and the sample acquisition module 8; the photoelectric detection module comprises a photoelectric detector 4 and a transimpedance amplifier (TIA), the photoelectric detector 4 is configured to receive a signal of the reference path signal light having been transmitted through the Michelson interferometer 3 as the reference signal, before receiving an echo signal generated by the sample to be scanned as the reflection signal; and configured to generate a current signal after an interference between the reference signal and the reflection signal, before transmitting the current signal to the transimpedance amplifier (not shown in the figure); the transimpedance amplifier is configured to generate a voltage signal correspondingly according to the current signal (that is, amplifying the current signal and converting into the voltage signal which is more convenient to process), before transmitting the voltage signal to the sample acquisition module 8. To the reference signal and the reflection signal (that is, the echo signal reflected back by the sample), it is noted that, both the reference signal and the reflection signal are optical signals, and after the reference signal and the reflection signal interfere jointly in the photoelectric detector 4, a current is generated, and the current is then converted into the voltage signal having been amplified by the TIA (due to the sample acquisition module 8 and the data processing module 9 subsequently being able to process the voltage signal only), the voltage signal is received by the sample acquisition module 8.

In a plurality of specific embodiments, the system further comprises a control module 7, configured to control an active part of a whole system, in an embodiment, the control module 7 is configured to adjust a delay amount of the first optical delay line continuously adjustable and a delay amount of the second optical delay line continuously adjustable; and configured to control each of the optical switches to adjust a total optical delay amount; after determining a target delay amount, a plurality of optical switches are selected to generate a delay most matching the target delay amount, further by both the delay amount of the first optical delay line continuously adjustable, together with the delay amount of the second optical delay line continuously adjustable, a relative delay amount between two paths is locked accurately to the target delay amount; as well as controlling the two-dimensional rotating mirror 51.

By switching the optical delay amounts of different reference paths in combination with a surface scanning of the two-dimensional rotating mirror 51, a three-dimensional image of the sample is finally generated by a data processing module 9.

A working principle of the present application is as follows:
the optical fiber couples the detection light emitted by the light source 1 to the optical input port of the optical chip 2, and the light splitter divides the light in the waveguide into a reference path light and a sample path light, wherein both paths have the optical delay lines arranged, which are fine adjustable, however, delays generated by the two paths alone are not enough to meet the relative delay amount required by the system, which is relatively large, therefore it is possible to turn on/off a switch in the reference path, to select a path having a delay amount matching an ideal delay amount the most (or it is possible to control the switch to superimpose the delays of multiple paths to meet such a condition), and after determining the delay matching with the optimal delay amount most, the delay amount is locked into a target value through two paths of optical delay lines at a front end, which are fine adjustable. Through the present combination mode, a large-scale adjustable delay amount can be achieved, and a final relative delay amount of two paths can be arbitrarily controlled from zero, which is able to improve greatly an axial resolution and an imaging accuracy. The sample path signal having been regulated by the delay line is coupled to the optical fiber through the FA, then a collimating lens collimates a light beam before guiding the light beam to a sample to be scanned (such as an eyeball) through the two-dimensional rotating mirror 51, so as to implement a two-dimensional slice scanning at a specific depth of the sample. The detection light reflected by the sample and the reference light from the fiber are received by the photoelectric detector 4 after passing through a Michelson beam instrument. The photoelectric detector 4 outputs the current signal generated after the two signals having been interfered, and the current signal is amplified into the voltage signal through the TIA. By changing the relative delay amount between the reference path signal and the sample path signal in the optical chip 2, obtaining information at different depths of the sample is achieved, after combining with scanning by the two-dimensional rotating mirror 51, a fine three-dimensional image of the sample is finally output by the data processing module 9. The present application has a plurality of advantages including: on one hand, by replacing an external mechanical displacement system with the photonic integrated chip, it is possible to reduce a volume, quality and cost of a system, but increase a reliability of the system; on another hand, due to a relatively low thermo-optical coefficient of the silicon nitride, it is difficult to achieve an adjustable optical delay amount in a range of dozens of millimeters by using a single delay line. Further, the present application, by adopting a single-order sub-millimeter-level continuous adjustable delay line, and cooperating together with a fixed delay line, achieves an optical delay amount continuously adjustable in an ultra large range with a precision of a photonic integrated device based on a silicon nitride platform; and on another hand, a relative delay amount between two outlets of the chip can be adjusted from zero.

For ease of understanding, in the present embodiment, a specific implementation process of the system stated above is further described here in combination with a specific application scenario system, shown as FIGS. 1-3, comprising specifically: a time domain optical coherence tomography (TD-OCT) system based on a photonic integrated chip, wherein the system comprises a light source 1, an optical chip 2, a Michelson interferometer 3, a two-dimensional rotating mirror 51, a scanning element 5, a photoelectric detector 4, a sample acquisition module 8, a control module 7, and a data processing module 9.

In the present embodiment, the light source 1 is a low-coherence broadband light source with a center frequency of 1060 nm, and is typically a super-radiation light-emitting diode with a bandwidth of 70 nm.

The light source 1 stated above achieves a high-efficiency coupling between the optical fiber and the optical chip 2 through an optical fiber in a manner of an edge coupler or a lens coupling. The optical chip 2 is composed by a substrate, a buried oxide layer, a silicon nitride waveguide layer, and a metal electrode, comprising a light input optical port (i.e. the first edge coupler 21), a light splitter, two optical delay lines arranged symmetrically on a reference path and a sample path, which are finely adjustable, an N+1-level cascaded optical switch on the reference path, an N-level cascaded optical delay line having a fixed delay amount, and two optical output ports (i.e. the second edge coupler 22 and the third edge coupler 23)

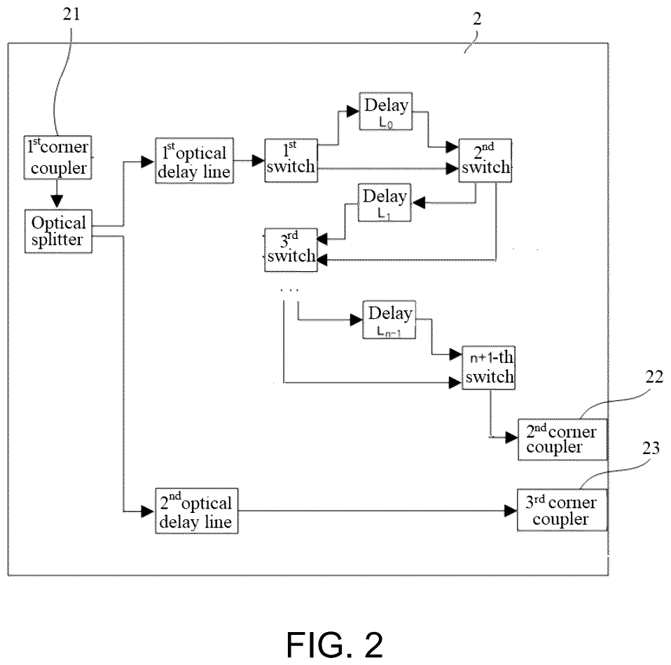

Referring to FIG. 2, an edge coupler on a left side of the optical chip 2 serves as an optical input port, coupling light in an optical fiber before converting into a guided wave in the silicon nitride waveguide, which is then divided into two paths through a 50:50 ADC light splitter, wherein an upper side channel is a reference path, and a lower side channel is a sample path. The reference path and the sample path each has an optical delay line of a spiral structure continuously adjustable arranged respectively and symmetrically. Specifically, the first optical delay line and the second optical delay line shown in FIG. 2 are completely a same. Both optical delay lines in two paths have a length of 40 cm, it is possible to adjust the delay amount through the thermo-optic effect of the silicon nitride, and a maximum delay amount adjustable of the two paths is about 500 μm equivalently. However, such a small delay amount cannot meet the imaging depth in the millimeter level in an OCT application. Therefore, the embodiments of the present application further introduce a delay in the reference path to adjust the relative delay amount of the two paths of signals.

The light in the reference path passes through the first optical delay line and then passes through the link formed by the N-level optical switch and the fixed optical delay line. The optical switch is a 2×2 type ADC. One of two output ports of the ADC is input into a designed optical delay path, before being connected to one input port of an ADC in a next level, and another output port is directly connected to another input port of the ADC in the next level through a straight waveguide (having an extremely small delay amount). Similarly, selecting a channel of a switch of the ADC is controlled by the thermo-optic effect of the silicon nitride waveguide. Wherein the optical delay line of each level has a fixed but different optical delay amount, and a length of the optical delay line on each level grows exponentially, L1=L, L2=2L, L3=4L, . . . , Ln=2ⁿ⁻¹L. In order to obtain a total delay amount of more than 10 mm, a fixed delay line selected by a five-level switch (n=5) is arranged in the present embodiment. Specifically, the delay amount introduced by a first-level fixed delay length after a reference arm first-level switch should be less than or equal to a maximum delay amount of 500 μm that a previous level adjustable delay line can generate. Therefore, the delay length corresponding to each level is respectively as follows: L1=400 μm, L2=800 μm, L3=1600 μm, L4=3200 μm, L5=6400 μm, thus a path of delay matching a desired delay amount may be selected by turning on/off a switch of the reference path (or the switch may also be controlled to superimpose multiple delays to meet the condition). In an embodiment, when the target delay amount is 900 μm, it should turn off a second switch to generate a delay of 800 μm, while all other switches should be turned to a straight-through state; and when the target delay amount is 2500 μm, both the second switch and a third switch in FIG. 2 should be switched at a same time to obtain a fixed delay amount of 2500 μm, while other switches are turned to the straight-through state. After determining the delay matching most with the desired delay amount, through the first optical delay line and the second optical delay line at the front end, the relative delay amount of the two paths is accurately locked to the target value. In a design of the present embodiment, the adjustable range of the relative delay amount between two paths is 0-12.9 mm, while a minimum step is not limited theoretically.

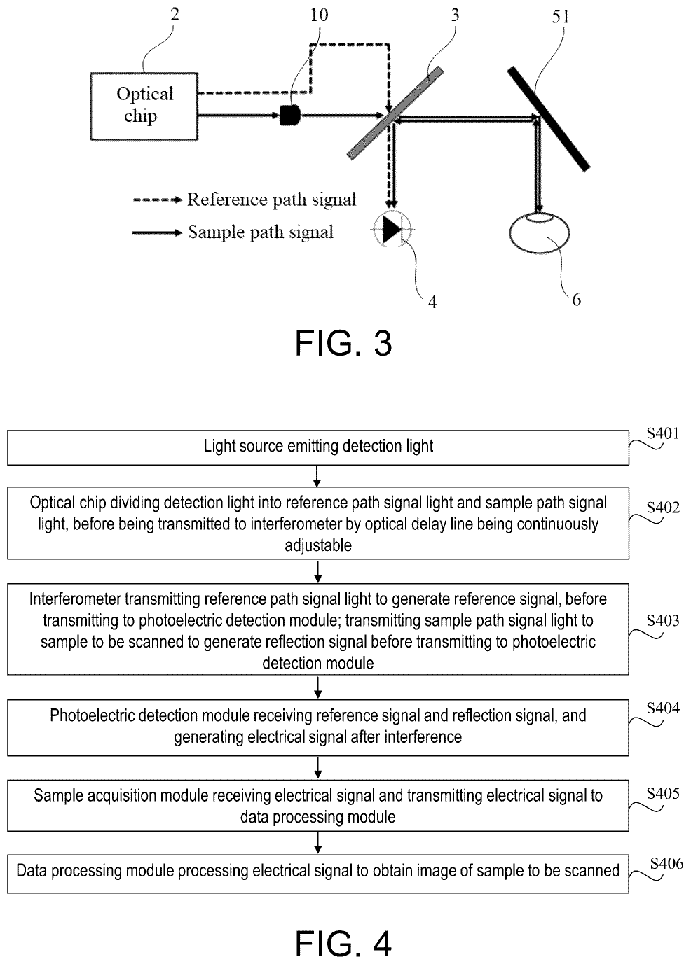

Two paths of the optical chip 2 are coupled to the bi-channel FA through the second edge coupler 22 and the third edge coupler 23. Referring to FIG. 3, a sample path signal from the optical chip 2 is collimated by the fiber collimator 10. The reference path signal light and the sample path signal light are then both transmitted through the optical fiber to the Michelson interferometer 3, wherein the reference path signal is received by the photoelectric detector 4 after passing through the Michelson interferometer 3, and the sample path signal is directed to the scanning element 5 after passing through the Michelson interferometer 3.

The scanning element 5 comprises a two-dimensional rotating mirror 51. The two-dimensional rotating mirror 51 guides the light coming from the Michelson interferometer 3 before being collimated to the sample to be detected, that is, the eye 6, and completes an extraction of the information on a specific depth level of a specific sample through the scanning of the two-dimensional rotating mirror 51.

The photoelectric detection module comprises a photoelectric detector 4 and a transimpedance amplifier (TIA). Wherein the photoelectric detector 4 receives the reference signal of the reference path in the optical chip 2, and the reflection signal generated by the sample to be scanned, before generating a current signal after an interference between the reference signal and the reflection signal. While the TIA amplifies the current signal into a voltage signal which is easy to process.

The control module 7 controls an active part of a whole system, including controlling the delay amount of both optical delay lines being finely adjustable in two paths in the optical chip 2, controlling the switch in each level of the reference path, controlling the two-dimensional rotating mirror 51, and more.

By switching the optical delay amounts of different reference paths, in combination with surface scanning of the two-dimensional rotating mirror 51, a three-dimensional image of the sample is finally generated by the data processing module 9. In addition, in the prior art, there are basically two methods to change the optical delay amount, one is adopting a light splitter to divide a sampling light beam into a plurality of sampling light beams, then changing an optical path of each of the plurality of sampling light beams to generate a time delay between the plurality of sampling light beams, followed by transmitting the plurality of sampling light beams to a sample to be detected through an output port, then the output port receives a plurality of reflection signals returned from the sample; the reflection signals are then interfered with the reference signal before obtaining information at different physical positions of the sample; another is adopting a plurality of optical switches having been cascaded, and each level has a delay line arranged, thus it is possible to decide a light in a waveguide shall be delayed or not by turning on/off an optical switch; each level has a different delay amount arranged, thus by the optical switch turns on/off a delay amount required, an action of adjusting the optical delay line is achieved. Wherein the optical switch is realized based on an electro-optical effect of a lithium niobate waveguide, while the delay line in the waveguide adopts a silicon nitride waveguide to obtain low transmission loss. In the first method, since the light splitter is a passive device, which is not adjustable, an imaging precision is limited by a number of the plurality of sampling light beams and a relative delay amount between different paths. A main defect of the second method is that, only a plurality of discrete specific delay amounts can be selected, instead of really achieving a continuously adjustable optical delay, also a technology of a large-scale lithium niobate combining with the silicon nitride waveguide is not mature, while the optical delay amount in both methods can only take a plurality of discrete values, instead of being able to be continuously adjustable and a high-precision.

Based on the time domain coherence tomography system based on the photonic integrated chip, shown as FIG. 4, the present application further provides a time domain coherence tomography method based on a photonic integrated chip, comprising:
S401, a light source emitting a detection light;
S402, an optical chip dividing the detection light into a reference path signal light and a sample path signal light, and transmitting the reference path signal light and the sample path signal light to an interferometer by an optical delay line continuously adjustable respectively;
S403, the interferometer transmitting the reference path signal light to generate a reference signal, then transmitting the reference signal to a photoelectric detection module; and transmitting the sample path signal light to a sample to be scanned to generate a reflection signal, then transmitting the reflection signal to the photoelectric detection module;
S404, the photoelectric detection module receiving the reference signal and the reflection signal, and generating an electrical signal after an interference;
S405, a sample acquisition module receiving the electrical signal and transmitting the electrical signal to a data processing module;
S406, the data processing module processing the electrical signal to obtain an image of the sample to be scanned.

While the embodiments of the present application have been described in detail above, it will be apparent to those skilled in the art that various modifications and variations can be made to the embodiments. It should be understood, however, that such modifications and variations are within the scope and spirit of the present application as set forth in the claims. Moreover, the present application described herein is capable of other embodiments and of being practiced or of being carried out in various ways.

What is claimed is:

1. A time domain coherence tomography system based on a photonic integrated chip, comprising a light source, an optical chip, an interferometer, a photoelectric detection module, a sample acquisition module and a data processing module, wherein the light source is connected to the interferometer by the optical chip, while both a reference path and a sample path of the optical chip have an optical delay line arranged respectively, and the optical delay lines are continuously adjustable;
the light source is configured to emit a detection light;
the optical chip is configured to divide the detection light into a reference path signal light and a sample path signal light, and transmit the reference path signal light and the sample path signal light to the interferometer by the optical delay lines respectively;
the interferometer is configured to transmit the reference path signal light to generate a reference signal, and transmitting the reference signal to the photoelectric detection module; and configured to transmit the sample path signal light to a sample to be scanned to generate a reflection signal, and transmitting the reflection signal to the photoelectric detection module;
the photoelectric detection module is configured to receive the reference signal and the reflection signal, and generating an electrical signal after an interference;
the sample acquisition module is configured to receive the electrical signal and transmit the electrical signal to the data processing module;
the data processing module is configured to process the electrical signal to obtain an image of the sample to be scanned;
the optical chip comprises an optical input port formed by a first edge coupler, a light splitter, a reference path, a sample path, a first output port formed by a second edge coupler, and a second output port formed by a third edge coupler; the reference path and the sample path having an first optical delay line which is continuously adjustable and a second optical delay line which is continuously adjustable arranged respectively and symmetrically;
after having been input by the first edge coupler, the detection light is divided into the reference path signal light and the sample path signal light by the light splitter, while the reference path signal light is output by the first output port, and the sample path signal light is output by the second output port;
the optical chip comprises a silicon substrate, a buried oxide layer, a silicon nitride waveguide layer, and a metal electrode;
the optical delay line is of a spiral structure; and/or the reference path of the optical chip further has a link arranged, the link is formed by an N+1-level cascaded optical switch and an N-level cascaded optical delay line, while an optical delay amount of each level of the optical delay line is fixed but different; wherein N is a positive integer;
the reference path signal light passes through the first optical delay line and passes through the link, and being output by the first output port;
the sample path signal light passes through the second optical delay line, and then being output by the second output port directly;
after determining a delay matching most with a desired delay amount, through the first optical delay line and the second optical delay line at a front end, a relative delay amount of the two paths is accurately locked to a target value.

2. The system according to claim 1, wherein a length of the optical delay line on each level of the N-level cascaded optical delay line with a fixed delay amount growing exponentially, while a delay amount of a next-level optical delay line is greater than a delay amount of a previous-level optical delay line; and/or the fixed delay amount of a first-level optical delay line is less than or equal to a maximum delay amount of the optical delay line.

3. The system according to claim 1, wherein the first output port and the second output port of the optical chip are coupled through a bi-channel optical fiber array, and the sample path signal light is collimated by an optical fiber collimator.

4. The system according to claim 1, wherein the optical switch is a 2×2 type adiabatic directional coupler or a multimode interferometer.

5. The system according to claim 1, wherein further comprising a control module, configured to adjust a delay amount of the first optical delay line and a delay amount of the second optical delay line; and configured to control the optical switch on each level to adjust a total optical delay amount; after determining a target delay amount and selecting a plurality of optical switches to generate a delay most matching the target delay amount, together with the delay amount of the first optical delay line and the delay amount of the second optical delay line, a relative delay amount between two paths is locked accurately to the target delay amount.

6. The system according to claim 1, wherein the light source is coupled to the optical chip through an optical fiber in a coupling manner of a coupler or a lens.

7. The system according to claim 1, wherein the photoelectric detection module is arranged between the interferometer and the sample acquisition module;
the photoelectric detection module comprises a photoelectric detector and a transimpedance amplifier;
the photoelectric detector is configured to receive a signal of the reference path signal light having been transmitted through the interferometer as the reference signal, and receive an echo signal generated by the sample to be scanned as the reflection signal; and configured to generate a current signal after an interference between the reference signal and the reflection signal, and transmit the current signal to the transimpedance amplifier;
the transimpedance amplifier is configured to generate a voltage signal correspondingly according to the current signal, and transmit the voltage signal to the sample acquisition module.

8. The system according to claim 1, wherein further comprising a scanning element, and the scanning element comprises a two-dimensional rotating mirror;
the scanning element is configured to guide the sample path signal light emitted by the interferometer to the sample to be scanned by the two-dimensional rotating mirror, so as to generate a plurality of echo signals at a plurality of different positions of the sample to be scanned, and completing extracting an information on a specific depth level of the sample to be scanned.

9. The system according to claim 1, wherein the light source is a low coherence broadband light source; and/or the interferometer is a Michelson interferometer.

10. A time domain coherence tomography method based on a photonic integrated chip, wherein comprising:
emitting a detection light by a light source;
dividing a detection light into a reference path signal light and a sample path signal light by an optical chip, and transmitting the reference path signal light and the sample path signal light to an interferometer by an optical delay line which is continuously adjustable respectively;
transmitting the reference path signal light to generate a reference signal by the interferometer, then transmitting the reference signal to a photoelectric detection module; and transmitting the sample path signal light to a sample to be scanned to generate a reflection signal, then transmitting the reflection signal to the photoelectric detection module;
receiving the reference signal and the reflection signal by the photoelectric detection module, and generating an electrical signal after an interference;
receiving the electrical signal by a sample acquisition module and transmitting the electrical signal to a data processing module;
processing the electrical signal to obtain an image of the sample to be scanned by the data processing module;
the optical chip comprises an optical input port formed by a first edge coupler, a light splitter, a reference path, a sample path, a first output port formed by a second edge coupler, and a second output port formed by a third edge coupler; the reference path and the sample path having an first optical delay line which is continuously adjustable and a second optical delay line which is continuously adjustable arranged respectively and symmetrically;
after having been input by the first edge coupler, the detection light is divided into the reference path signal light and the sample path signal light by the light splitter, while the first output port outputs the reference path signal light, and the second output port outputs the sample path signal light;
the optical chip comprises a silicon substrate, a buried oxide layer, a silicon nitride waveguide layer, and a metal electrode;
the optical delay line is of a spiral structure; and/or the reference path of the optical chip further has a link arranged, the link is formed by an N+1-level cascaded optical switch and an N-level cascaded optical delay line, while an optical delay amount of each level of the optical delay line is fixed but different; wherein N is a positive integer;
the reference path signal light passes through the first optical delay line and passes through the link, and being output by the first output port;
the sample path signal light passes through the second optical delay line, and then being output by the second output port directly;
after determining a delay matching most with a desired delay amount, through the first optical delay line and the second optical delay line at a front end, a relative delay amount of the two paths is accurately locked to a target value.
